(19) United States
(12) Patent Application Publication
(10) Pub. No.: US 2021/0369840 A1
(43) Pub. Date: Dec. 2, 2021

(54) METHODS OF USE & COMPOSITIONS OF IBD, IBS, & ANTINEOPLASTIC MICROBIAL THERAPEUTICS

(71) Applicant: John Malcolm Hall Gregg, Princeton, NJ (US)

(72) Inventor: John Malcolm Hall Gregg, Princeton, NJ (US)

(21) Appl. No.: 16/326,465

(22) PCT Filed: Aug. 20, 2017
(86) PCT No.: PCT/US2017/047706

§ 371 Date: Feb. 19, 2019

(51) Int. Cl. A61K 39/39; A61K 39/00; A61K 39/114; A61K 39/08; A61K 39/02; A61K 39/108; A61K 39/112; A61K 39/05; A61K 39/09; A61K 39/07; A61K 39/085; A61P 37/04; A61P 1/14

(52) U.S. Cl. CPC A61K 39/39; A61K 2039/6087; A61K 39/114; A61K 39/08; A61K 39/105; A61K 39/0258; A61K 39/107; A61K 39/0275; A61K 39/025; A61K 39/0283; A61K 39/05; A61K 39/09; A61K 39/092; A61K 39/07; A61K 39/085; A61K 39/0208; A61P 37/04; A61P 1/14; A61K 2039/55544; A61K 2039/585; A61K 39/0011

(57) ABSTRACT

This invention relates to the medical use of pathogen associated molecular pattern (PAMP) displaying immunostimulatory microbial immuno-adjuvants [MIAs], as therapeutics when used in combination with check point inhibitors to treat various types of cancer and to methods of treatment which involve treating a subject with these compounds and compound mixtures and process methods for identifying and optimally composing them for their therapeutic use in cancer treatment. It also relates to the use of inhibitors of these PAMP displaying immunostimulatory microbial immuno-adjuvants [MIAs], as therapeutics when used to treat inflammatory bowel disease (IBD) including Crohn's Disease and ulcerative colitis and irritable bowel syndrome (IBS) and process methods for identifying and optimally composing them for their therapeutic use in IBD and IBS.

### CROSS-REFERENCE TO RELATED APPLICATIONS

This application is a national phase entry under 35 U.S.C. § 371 of International Application PCT/US2017/047706, filed Aug. 20, 2017, which claims the benefit under 35 U.S.C. § 119(e) of U.S. Provisional Application No. 62/494,763 filed Aug. 20, 2016, all of which are herein incorporated by reference in their entireties.

### BACKGROUND

Immune tolerance prevents the eradication of cancer in many human sufferers of these neoplastic diseases because their own immune system has lost the ability to distinguish between neoplastic and non-neoplastic cells. A new class of drugs called “checkpoint inhibitors” which include PD-1 (PD-L1) and CTLA-4 targeted pathways were developed, first in melanoma, and later for other types of cancer, to restore a vigorous immune response to tumor cells and neoplastic tissue. The drugs include, Yervoy (ipilimumab), Opdivo (nivolumab) and Keytruda (pembrolizumab).

Prior to the introduction of checkpoint inhibitors, the prognosis of all metastatic melanoma patients was very poor. Following the introduction to these drugs the response and enhanced survival of one quarter to one third of patients treated was dramatically improved. However, a large majority of metastatic melanoma patients have not significantly responded to these therapies for reasons that are not fully known.

Additionally, the response to these drugs in other types of cancer which have traditionally been more immunologically responsive, like renal cell carcinoma, lung cancer, breast cancer, bladder cancer, and prostate cancer have not demonstrated the same level of responsiveness to these drugs as patients with metastasized melanoma.

It is hypothesized that in addition to inhibiting the checkpoint systems in immune cells, that it is necessary to stimulate immune systems cells to provoke and coordinate attack against neoplastic cells in order to increase the percentage of responding metastatic melanoma patients to these therapies and also to extend their benefits to other types of cancer in therapeutically meaningful percentages of responding patients.

The degree of effectiveness of checkpoint inhibitors could potentially be enhanced by the concomitant administration of partner drugs in defined regimens that would enhance the activation of the key types of immune cells, like T-cells and macrophages, and improve their recognition of target neoplastic cells. A number of different types of co-adminstered drugs are being evaluated to see if any of them would have beneficial sensitizing effects on checkpoint inhibitors, however, some of the most promising effects appear to come from modulating the microbiomes of the gastro-intestinal systems of afflicted sufferers.

Published papers have shown beneficial results in expanding the effectiveness of checkpoint inhibitors in murine systems of melanoma with either administration of specific strains Bifidobacteria or injection with antibodies raised against those bacteria or lipopolysaccharides (LPS) from those specific strains. Similar results were obtained in a system that involved maintaining the presence of Bacteroides thetaiotamicron and Bacteroides fragilis. It is therefore thought that there is a gastrointestinal microbiome approach of stimulating the immune system to attacking cancer cells following the administration of checkpoint inhibitors that mitigate the brakes on cells that would otherwise be anergic and tolerant of the neoplastic cells.

Bacterial species in the gastrointestinal tract are generally symbiotic with their human holobiont hosts and aid in food digestion in a mutualistic relationship as long as they are maintained in their appropriate enclosed GI compartment. However, there is always a possibility that if the physical barrier in the mucosa or epithelium of the GI tract is breached that these bacteria could travel to other compartments via systemic circulation and overgrow to become pathogenic.

Treg immune cells in the GI system therefore regularly sample bacteria cells within the GI system that have the potential to breach barriers and overgrow if released into other compartments or the systemic circulatory lymphatic or cardiovascular networks. Those components of the bacterial cells and their metabolic products that have the greatest potential to elicit a vigorous immunological response have specific Pathogen Associated Molecular Patterns (PAMPs) [also sometimes known as “Microbial Associated Molecular Patterns (MAMPs)].

PAMPs are molecules associated with groups of pathogens that are recognized by cells of the innate immune system. These molecules can be referred to as small molecular motifs conserved within a class of microbes. They are recognized by Toll-like receptors (TLRs) and other pattern recognition receptors (PRRs). A vast array of different types of molecules can serve as PAMPs, including glycans and glycoconjugates.

PAMPs activate innate immune responses, protecting the host from infection, by identifying some conserved nonself molecules. Bacterial lipopolysaccharides (LPSs), endotoxins found on the cell membranes of bacteria, are considered to be the prototypical class of PAMPs. LPSs are specifically recognised by TLR4, a recognition receptor of the innate immune system. Although the term “PAMP” is relatively new, the concept that molecules derived from microbes must be detected by receptors from multicellular organisms has been held for many decades, and references to an “endotoxin receptor” are found in much of the older literature. Exotoxins released from lysed bacterial cells are also potent immunological activators and are a source of PAMPs that can be recognized by PRRs.

Escaping the GI compartment via breech in mucosal and epithelial barriers could result in dangerous bacteremias and spread to other tissue compartments. Many of the bacterial species in the GI compartment are anaerobic. While some of these are facultative and can switch to aerobic respiration pathways if necessary, many others are obligate or near obligate anaerobes. These bacteria after breaching the barriers to the GI compartment and being exposed to oxygen become reactive oxygen species (ROS) producers upon escape, manufacturing hydrogen peroxide, nitric oxide, IL-6 and TNF alpha. These molecules, like PAMPs, have an immunostimulatory effect on macrophages and a variety of regulatory immune cells and can potentiate these cells that are simultaneously treated with checkpoint inhibitors to attack cancer cells. PAMPs on these cells when occupying non-GI luminal, mucosal, and epithelial compartments indicate the presences of these cells in places where they are not supposed to be located and also induce an immunostimulant and immuno-adjuvant response.

Bacteroides fragilis is an excellent model for this oxygen exposure model. Previously classified as a “strict anaerobe,” this mammalian commensal has since been found to grow in and benefit from nanomolar concentrations of oxygen. In addition, even though it cannot replicate in room air, this organism is capable of mounting a strong response to aeration, including the production of numerous catalases and other PAMP displaying ROS-scavenging enzymes that contribute to its impressive, though transient aerotolerance. Components of these enzymes that display PAMPs are also included in the present invention and adjuvant ingredients to stimulate immune cells that have been treated with checkpoint inhibitors to attack cancer cells.

While the present invention exploits the potential of therapeutics displaying PAMPs to act as microbial immuno-adjuvants (MIAs) in concert with checkpoint inhibitors, it also exploits similarly bacterial derived compound dampen response to PAMPs that result in IBD & IBS and their related flare symptomatology. There is a strong analogy to the pathophysiological mechanism of flares of Crohn's Disease and ulcerative colitis and inflammation and diarrhea in IBS to the concept of allergy and allergic response to antigens by the immune system.

The inflammatory response in the case of IBD and IBS is related to exposure to increased levels of specific bacterial PAMPs rather than exposure to antigens presented by the specific food that is consumed. If mammals are regarded as holobionts that consist of the host mammal and all of the microbiota living in or colonizing that host, then the exposure to specific PAMPs is related to their relative abundance and specific points in time. Food can be regarded as one type of prebiotic because it is delivering nutrients to both the host and the symbiotic and parasitic microbiota present in the various microbiomes resident in the host. The type of food consumed in a particular meal will provide a unique nutrient base for the microbiota in the different microbiomes of the gastrointestinal system. These bacteria, located in the lumen and the mucosal and epithelial surface of the GI organs will respond to specific nutrient offerings that they can utilize by metabolizing nutrients their species and strain can metabolize and will reproductively expand in response to favourable nutrient sources. This expansion differentially increase the relative abundance of specific PAMPs which may elicit different responses in different hosts, who have all evolved over their lifetimes to react differently based on their prior microbial exposure and barrier breach history. This is analogous to differential allergic response by different individuals to the same antigens present in different foods. Here, the food is not having a direct effect based on its antigens. It is having an indirect effect based on favoring the expansion of different microbial species that express their own PAMPs capable of eliciting different immunological responses.

The differential effect of bacterial compositional diversity and relative bacterial species and strain abundances upon exposure to prebiotic food nutrient abundance explains why historically there has been some effect, but not a highly dramatic or curative one the administration of antibiotics to treat IBD and IBS. If the immunostimulatory effects eliciting an inflammatory response happen to be engendered by, among others, bacterial species producing PAMPs by the species sensitive to the antibiotic, then the relative decrease in abundance of those species and their PAMPs could have a positive impact on inflammatory symptomatology. If the immunostimulatory effects eliciting an inflammatory response happen to be engendered by bacterial species producing PAMPs other than those sensitive to the antibiotic, then the relative decrease in abundance of those species and their PAMPs would be unchanged or possibly increased by changes in relative compositional abundance which could thus have either a neutral or negative impact on inflammatory symptomatology.

These effects, some which might be countervailing with relative abundances of different species, would be magnified by cocktails of broad spectrum antibiotics which would have impacts on various microbiome microbiota compositional diversities and relative abundances. However, antibiotics are not administered chronically in humans so that the end of antibiotic treatment would put an end to exogenous selective pressure on the microbiota, some of which would rebound because they maintained their colonization presence through biofilms or were re-introduced from other compartments or the consumption of spores. These changes in relative abundances due to the introduction of antibiotics and prebiotics (including food), would have substantive impacts on the levels of specific immunostimulatory PAMPs exposed to the immune systems of individuals. The patterns of response would be personalized so that treatment would either be generalized through treatment to dampen response to all PAMPs or customized for treatment to lower the relative abundance of specific PAMPs that induce an allergic like inflammatory response by selectively modulating/reducing the abundance of these specific bacterial strains that have been identified as relevant for that individual.

### SUMMARY OF THE INVENTION

The present invention relates to methods of treating and/or reducing the incidence of various types of cancer and neoplastic disease by administering to a subject in need thereof a therapeutically effective amount of live bacterial therapeutics with strongly immune activating PAMPs or enteric and/or parenteral administration of lipopolysaccharides that exhibit these PAMPs, attenuated endotoxins or exotoxins containing PAMPs, or infusion of antibodies raised against these PAMPs.

The present invention includes both individual PAMPs from specific bacterial species and selected strains of those species as well as mixtures of PAMPs from a variety of species in the form of administered polysaccharides that have the impact of providing a greater immunostimulatory effect on cells regulated by checkpoint inhibitors with the potential to attack tumor cells. These PAMPs and PAMP mixtures act as Microbial Immuno-adjuvants (MIAs) that are Microbial Immuno Stimulant (MIS) compositions that are co-administered with checkpoint inhibitors to enhance their effectiveness in terms of percentage of patients responding to checkpoint inhibitor therapy and range of responsive cancer types.

The methods of the present invention also include treating a disease associated with neoplastic cell growth of immunological responsive cancers that includes, but are not limited to, metastatic melanoma, lung cancer, renal cancer, breast cancer, colorectal cancer, gastric cancer, prostate cancer, bladder cancer, ovarian cancer, hematologic malignancies, pancreatic cancer, brain cancer, head & neck cancer, esophogeal cancer, hepatic cancer, and sarcomas.

In a specific embodiment, the toxins exhibiting PAMPs are produced by one or more organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In another embodiment, the administered companion regimen to checkpoint inhibitors for cancer is a mixture of two or more toxins, attenuated toxins, and regions of toxins displaying immunostimulatory PAMPS selected from those produced by specific strains of bacterial organisms selected from the group consisting of Fusobacterium species (including F. nucleatum and F. necrophorum), Helicobacter species (including Helicobacter Pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In another embodiment, the administered companion regimen to checkpoint inhibitors for cancer is a mixture of two or compounds produced by hydrogen peroxide, nitric oxide, and reactive oxygen species (ROS) producing bacterial cells displaying immunostimulatory PAMPS selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In another embodiment the PAMPs are produced by therapeutically administered (enteral and parenteral formulations) lipopolysaccharides (LPS) derived from cellular components of bacteria selected from the group consisting of Fusobacterium species (including F. nucleatum and F. necrophorum), Helicobacter species (including Helicobacter Pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In another embodiment, the administered companion regimen to checkpoint inhibitors for cancer is a mixture of lipopolysaccharides, and/or regions of lipopolysaccharides displaying immunostimulatory PAMPS derived from those produced by two or more specific strains of bacterial organisms selected from the group consisting of Fusobacterium species (including F. nucleatum and F. necrophorum), Helicobacter species (including Helicobacter Pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In another embodiment, the administered companion regimen to checkpoint inhibitors for cancer is a mixture of antibodies raised against two or more toxins, attenuated toxins, and regions of toxins displaying immunostimulatory PAMPS selected from those produced by specific strains of bacterial organisms selected from the group consisting of Fusobacterium species (including F. nucleatum and F. necrophorum), Helicobacter species (including Helicobacter Pylon), Prevotella species, Bacteroides species (including B. thetaiotamicron and B. fragilis), Clostridium species (including C. difficile, C. perfringens, and C. botulinum), Porphymonas species, Escherichia species (including E. co/i), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In another embodiment, the administered companion regimen to checkpoint inhibitors for cancer is an immunological mixture is of antibodies raised against two or more lipopolysaccharides or regions of lipopolysaccharides displaying immunostimulatory PAMPS selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

The methods of the present invention also include treating bowel disease including inflammatory bowel disease (IBD) comprised of Crohn's Disease and ulcerative colitis as well as irritable bowel syndrome (IBS)

As IBD and IBS arise from an exaggerated immunological response, similar to allergies, coming from exposure to specific PAMPs from LPS, endotoxins, and exotoxins derived from people's microbial (bacteria, fungi, viruses, protozoa) microbiomes that are periodically amplified by organismal compositional abundance expansion in response to prebiotic and nutritional inputs, including food, these responses can be blocked, mitigated, or attenuated by the therapeutic administration of selected toxin compounds that down-regulate the immunological response to PAMPs.

In a specific embodiment, the administered therapeutic is a toxin or mixture of two or more toxins, attenuated toxins, capable of down-regulating the IBS or IBD immunostimulatory PAMPs, with the toxin or toxins being selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

### DETAILED DESCRIPTION OF THE INVENTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

Where appropriate, any one or more of the other active agents may be in the form of a pharmaceutically acceptable salt.

Suitable pharmaceutically acceptable salts include, but are not limited to, salts of pharmaceutically acceptable inorganic acids such as hydrochloric, sulfuric, phosphoric, nitric, carbonic, boric, sulfuric, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

It is intended that the aspects and embodiments of this invention encompasses LPS, attenuated toxin, and/or any other active agent in all solid forms, including amorphous forms, as well as crystalline forms, and polymorphs thereof.

Throughout this specification the term ‘in combination’ means that one or more other actives are both administered to the patient over the same period of treatment. They may be administered together, i.e. at the same time. In this case they may be administered in a single formulation, (e.g. as a single tablet or capsule or sachet) or in separate formulations administered simultaneously or nearly simultaneously. Alternatively, they may be administered at separate times of day.

The combinations of the invention provide benefits which are at least additive compared to the use of either agent alone. In many embodiments, the combinations are something more than additive, e.g. synergistic, compared to the use of either agent alone.

The definition of the term ‘treatment’ in this specification encompasses prophylaxis and prevention (i.e. reducing or eliminating the risk of contracting the disease). As well as meaning curing a person of the disease, ‘treatment’ also includes preventing the onset of symptoms, controlling (e.g. by slowing or eliminating) progression of disease, preventing the spread of the disease to other parts of the body and/or to other persons, reducing the spread of the disease and other facets of medical practice which will be readily understood by the person skilled in the art to fall within the meaning of the term ‘treatment’.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

### Formulations

For the above-mentioned therapeutic uses the dosage administered will, of course, vary with the compound employed, the mode of administration, the treatment desired and the disorder indicated.

The pharmaceutical compositions may be administered systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration, one or more active agents may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, one or more active agents may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration, one or more active agents may be administered as a sterile aqueous or oily solution. Parenteral formulations are particularly suitable for patients suffering from a severe infections. The person skilled in the art would be well aware of what differentiates a serious infection from a non-serious infection. By way of example, severe infections include those which render the patient unable to take the active agents orally, e.g. infections which render the patient unconscious, emetic, weak, delirious etc.

### Treatment of Cancer

The present invention includes MIA products associated with PAMPs and used in conjunction with check point inhibitors to treat cancers that include, but are not limited to, metastatic melanoma, lung cancer, renal cancer, breast cancer, colorectal cancer, gastric cancer, prostate cancer, bladder cancer, ovarian cancer, hematologic malignancies, pancreatic cancer, brain cancer, head & neck cancer, esophogeal cancer, hepatic cancer, and sarcomas. It also includes MIA products with tumor specific molecular patterns (TSMPs).

In the present invention, the administered companion regimen to checkpoint inhibitors for cancer is a mixture of two or more toxins, attenuated toxins, and regions of toxins displaying immunostimulatory PAMPS selected from those produced by specific strains of bacterial organisms selected from the group consisting of Fusobacterium species (including F. nucleatum and F. necrophorum), Helicobacter species (including Helicobacter Pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In the present invention, the administered companion regimen to checkpoint inhibitors for cancer is a mixture of two or compounds produced by hydrogen peroxide, nitric oxide, and reactive oxygen species (ROS) producing bacterial cells displaying immunostimulatory PAMPS selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In the present invention, the PAMPs are produced by therapeutically administered (enteral and parenteral formulations) lipopolysaccharides (LPS) derived from cellular components of bacteria selected from the group consisting of Fusobacterium species (including F. nucleatum and F. necrophorum), Helicobacter species (including Helicobacter Pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In the present invention, the administered companion regimen to checkpoint inhibitors for cancer is a mixture of lipopolysaccharides, and/or regions of lipopolysaccharides displaying immunostimulatory PAMPS derived from those produced by two or more specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In the present invention, the administered companion regimen to checkpoint inhibitors for cancer is a mixture of antibodies raised against two or more toxins, attenuated toxins, and regions of toxins displaying immunostimulatory PAMPS selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenfi, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

In the present invention, the administered companion regimen to checkpoint inhibitors for cancer is an immunological mixture is of antibodies raised against two or more lipopolysaccharides or regions of lipopolysaccharides displaying immunostimulatory PAMPS selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

### Treatment of IBD/IBS

In the present invention, the administered therapeutic is a toxin or mixture of two or more toxins, attenuated toxins, capable of down-regulating the IBS or IBD immunostimulatory PAMPs, with the toxin or toxins being selected from those produced by specific strains of bacterial organisms selected from the group consisting of Fusobacterium species (including F. nucleatum and F. necrophorum), Helicobacter species (including Helicobacter pylon), Prevotella species, Bacteroides species (including B. thetaiotamicron and B. fragilis), Clostridium species (including C. difficile, C. perfringens, and C. botulinum), Porphymonas species, Escherichia species (including E. coli), Vibrio species (including V. cholerae), Campylobacter species (including C. jejuni), Salmonella species (including S. enterica and serovar S. Typhi), Enterobacter species (including E. aerogenes and E. cloacae), Shigella species (including S. dysenteriae, S. flexneri, and S. sonnei), Bifidobacterium species (including B. infantis), Lactobacillus species (including hydrogen peroxide producing strains of L. acidophilus, L. jensenii, and L. catenaforme), Bacillus species (including B. subtilus and B. licheniformis), Peptococcus species, Peptostreptococcus species, Streptococcus species (including S. pyogenes), Actinomyces species, Staphylococcus species (including S. aureus), Enterococcus species (including Enterococcus faecalis), and Listeria species (including L. monocytogenes).

### Methods of Bioinformatics and Process of PAMP Expressing Product Development

The bioinformatics of the present invention employs a base of whole genome shotgun sequencing analysis of metagenomic data that uses iterative scanning of small motifs, including 12 amino-acid (36 bp) motifs, that are then compared for a comprehensive taxonomy against all 280,000 named organisms in public databases and are benchmarked against other pipelines (e.g., MetaPhlan, Phylosift, GOTTCHA and Kraken). These analyses enable the identification of the motifs in PAMPs through proteomic and transcriptomics by mapping genes encoding endotoxins and exotoxins to those molecules and the LPSs associated with different bacterial strains and can map near homologous TSMPs.

The present invention includes combinations of MIAs that are therapeutically beneficial in cancer and will be demonstrated as non-naturally occurring and involving inventive steps as contemplated in the intellectual property scheme described in Exhibit 1: Novel IP Regime: Patenting Microbial Ecologies.

Table 1 gives a summary of various bacterial organisms and in which clinical sites they are observed.

TABLE 1

| | Clinical sites of infection | | | | | |
|---|---|---|---|---|---|---|
| | | | | Soft | | Intra- |
| Organism | GI | Oral | Respiratory | tissue | Pelvic | abdominal |
| Gram-negative organisms | | | | | | |
| Bacteroides fragilis | +++ | | + | +++ | ++ | +++ |
| Bacteroides thetaiotamicron | +++ | | | ++ | | +++ |
| Campylobacter jejuni | +++ | | | + | | ++ |
| Clostridium botulinum | +++ | | | ++ | | |
| Clostridium difficile | +++ | | | + | | |
| Clostridium perfringens | +++ | | | + | | |
| Enterobacter aerogenes | +++ | | | | | +++ |
| Enterobacter cloacae | +++ | | | | | +++ |
| Escherichia coli | +++ | | | ++ | | +++ |
| Fusobacterium gonidiaformans | + | | | | ++ | |
| Fusobacterium necrophorum | + | + | +++ | ++ | + | ++ |
| Fusobacterium nucleatum | + | ++ | +++ | ++ | + | ++ |
| Helicobacter pylori | +++ | | | | | + |
| Parabacteroides distasonis | | | | ++ | | +++ |
| Porphyromonas asaccharolytica | ++ | | | ++ | | ++ |
| Porphyromonas gingivali | + | +++ | + | | + | + |
| Porphyromonas uenonis | + | | | | ++ | |
| Prevotella amnii | +++ | | | | +++ | |
| Prevotella bivia | +++ | | | + | +++ | + |
| Prevotella disiens | +++ | | | | +++ | |
| Prevotella melaninogenica | +++ | + | ++ | ++ | +++ | |
| Prevotella timonensis | +++ | | | + | +++ | |
| Megasphaera 1&2 | +++ | | | | | |
| Salmonella enterica | +++ | | | ++ | | +++ |
| Salmonella Typhi | +++ | | | ++ | | +++ |
| Shigella dysenteriae | +++ | | | | | +++ |
| Shigella flexneri | +++ | | | | | +++ |
| Shigella sonnei | +++ | | | | | +++ |
| Vibrio cholerae | +++ | | | + | | ++ |
| Gram-positive organisms | | | | | | |
| Actinomyces israelii | + | + | + | | + | |
| Actinomyces neuii ssp neuii | + | + | | + | + | + |
| Actinomyces odontolyticus | + | ++ | + | ++ | + | + |
| Actinomyces radingae | + | ++ | + | + | + | + |
| Actinomyces turicensis | + | ++ | + | ++ | ++ | ++ |
| Enterococcus faecalis | +++ | | | ++ | ++ | ++ |
| Listeria monocytogenes | +++ | | | | + | +++ |
| Peptostreptococcus anaerobius | + | | | | ++ | ++ |
| Staphylococcus aureus | + | +++ | +++ | +++ | | |
| Streptococcus pyogenes | + | ++ | +++ | ++ | | |

+, infrequent;
++, sometimes;
+++, frequently

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings, such as attached FIG. 1 Syntheses), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method for the treatment or prophylaxis of neoplastic cancerous disease comprising administering one or more active agents selected from the group consisting of one or more Pathogen Associate Molecular Pattern (PAMP) displaying compounds or pharmaceutically acceptable salts thereof, PAMP recognizing antibodies, PAMP displaying enteral and injectable lipopolysaccharides (LPSs), and mixtures thereof as microbial immuno-adjuvants to increase both the range of tumor types treated by check point inhibitors and the percentage of patients with a specific tumor type responding to check point inhibitor therapy.

2-8. (canceled)

9. The method of claim 1, wherein the cancerous disease is selected from the group consisting of metastatic melanoma, lung cancer, renal cancer, breast cancer, colorectal cancer, gastric cancer, prostate cancer, bladder cancer, ovarian cancer, hematologic malignancies, pancreatic cancer, brain cancer, head & neck cancer, esophogeal cancer, hepatic cancer, and sarcomas.

10. A method of using PAMP identifying small molecular motifs in bioinformatic analysis, the method comprising
(a) using data from both whole genome shotgun DNA sequencing and, transcriptomics and proteomics,
(b) identifying PAMP displaying products that can be recognized by pattern recognition receptors (PRRs) and serve as microbial immune-adjuvants (MIAs) in cancer treatment when used in conjunction with check point inhibitors.

11. The method of claim 10 wherein the PAMP displaying products identified in step (b) serve as microbial immune-adjuvants (MIAs) in cancer treatment when used in conjunction with check point inhibitors.

12. The method of claim 10 wherein the PAMP displaying products identified in step (b) are useful for immunological stimulation in the cancer setting,

13. The method of claim 12, wherein the PAMP displaying products concomitantly serve as a means of targeting PAMPs to be blocked, deleted, or downregulated by identified compounds to use in the treatment of inflammatory bowel disease (IBD) and inflammatory bowel syndrome (IBS).

14. A method comprising administering of one or more of a moiety selected from the group consisting of attenuated versions of identified endotoxins and attenuated versions of identified exotoxins, that display one or more PAMPs, to treat a condition selected from the group consisting of cancer, IBD, and IBS.

15. The method of claim 14 wherein the attenuated versions of identified endotoxins are selected from Botox (onobutulinumtoxiaA).

16. The method of claim 14 further comprising the administration of check point inhibitors to induce an immunostimulatory response in immune cells, and wherein the condition is cancer.

17. The method of claim 14 wherein the PAMPSs have close homologies to tumor-specific molecular patterns (TSMPs) and wherein the condition is cancer.

18. The method of claim 17 further comprising the administration of check point inhibitors to induce an immunostimulatory response in immune cells and to target and treat the cancer.

19. The method of claim 14, wherein the moiety blocks, deletes, or downregulates the immunostimulatory response in immune in the treatment of flares and symptomatology associated with IBD and IBS.
